(19) United States
(12) Patent Application Publication
(10) Pub. No.: US 2010/0296970 A1
(43) Pub. Date: Nov. 25, 2010

(54) Apparatus and Methods for Sanitizing Feet And/Or Footwear

(75) Inventors: Joseph Trimarco, Moriches, NY (US); Rachel Trimarco, Moriches, NY (US)

Correspondence Address: MOAZZAM & ASSOCIATES, LLC, 7601 LEWINSVILLE ROAD, SUITE 304, MCLEAN, VA, 22102, US

(73) Assignee: SANIHOME PRODUCTS INTERNATIONAL LLC, Center Moriches, NY (US)

(21) Appl. No.: 12/784,197

(22) Filed: May 20, 2010

Related U.S. Application Data
(60) Provisional application No. 61/180,785, filed on May 22, 2009.

(51) Int. Cl. A61L 2/18; A47K 3/022

(52) U.S. Cl. 422/37; 422/28; 422/292

(57) ABSTRACT

Apparatus and methods are disclosed for sanitizing feet and footwear. A sanitizing mat includes a base portion comprising a bottom surface and raised walls to create a reservoir. A sponge element containing a sanitizing solution sits within the reservoir. A surface element covers the top of the sponge element. The surface element is permeable, such that the resultant pressure of a user stepping on the sanitizing mat squeezes the sanitizing solution out of the sponge element and through the surface element to sanitize the feet or footwear of the user. The surface element comprises an abrasive material, such that debris from the user's feet or footwear may be loosened and left on the surface element. The user then steps off of the mat without carrying germs or bacteria on the soles of their feet or footwear.

This application claims priority to U.S. Provisional Patent Application Ser. No. 61/180,785, filed May 22, 2009, the content of which is hereby incorporated by reference herein in its entirety into this disclosure.

### BACKGROUND OF THE INVENTION

1. Field of the Invention

The present invention relates to sanitizing. More specifically, the present invention relates to mats with an integrated sanitizer to sanitize the soles of feet and/or footwear.

2. Background of the Invention

Throughout the day, people frequently come in contact with germs, such as bacteria, viruses, and other possibly harmful microorganisms. By holding railings, shaking hands, or even walking down the street, people pick up these germs and carry them along. While most bacteria are harmless, many disease-causing bacteria produce toxins that damage cells and cause illness. Viruses invade cells in the body, affecting the workings of the cell. Neither these, nor other microorganisms, are generally desirable to most people on their skin or their surrounding environment. Further, unlike most healthy people that generally harbor some microorganisms within their system, individuals having auto-immune deficiencies cannot tolerate this and are especially susceptible to even trace amount of microorganisms.

Realizing this, many people wish to disinfect or sanitize themselves and their environment. For instance, many people now carry small bottles of hand sanitizer wherever they go. This allows them to keep their hands clean, even after coming in contact with these railings, hands, etc. Sanitizers are substances that aim to reduce the number of microorganisms to a safe level. While conventional sanitizers may clean the person's hands, the environment still contains many other microorganisms. However, shortly after application of a sanitizer the person is back in contact with the microorganisms again by, for example, contact with a chair or other furniture in the household.

Thus, these current solutions do little about another source of the problem, germs in the household. Germs are constantly brought into the household through various vehicles including, for instance, on the soles of footwear. Therefore, even after washing one's hands entering a house, there is still the potential for coming in contact with more bacteria and viruses. Rarely does one think to wash hands after touching items in the household.

Regular door mats do little to solve the problem of germs coming into the household on the soles of shoes. These door mats generally include a rough material on their surface to brush the dirt off. However, much of the bacteria or other microorganisms remains alive on the soles and is brought into the household. Therefore, what is needed is a mat that sanitizes the soles of the footwear such that germs are not brought into the living or other desired protected area.

### SUMMARY OF THE INVENTION

The present invention provides a sanitizing mat for sanitizing the soles of footwear and feet. The sanitizing mat includes a sponge element containing a sanitizing solution. A surface element covers the top of the sponge element. The side and bottom surfaces of the sponge element and the sides of the surface element are enclosed by a base portion that also acts as a reservoir for the sanitizing solution. The surface element is composed of a material, such that when a user steps on the sanitizing mat, the sanitizing solution seeps out of the sponge element and through the surface element to sanitize the feet or footwear of the user. The surface element may be an abrasive material, such that debris from the user's feet or footwear may be loosened and left on the surface element. The user may then step off of the mat without carrying germs or bacteria on the soles of their feet or footwear.

Embodiments of the present invention further include a blotting portion. The blotting portion may be coupled to the sanitizing mat or may be separate from the sanitizing mat. The blotting portion soaks up excess sanitizing solution from the soles of a user's feet or footwear when the user steps off of the surface element onto the blotting portion. The blotting portion may be folded onto or placed onto the sanitizing mat to reduce evaporation of the sanitizing solution.

In one exemplary embodiment, the present invention is an apparatus for sanitizing. The apparatus includes a base portion having a reservoir, the reservoir formed by a bottom surface of the base portion and a plurality of side walls of the base portion, a sponge element coupled to the base portion, the sponge element being impregnated with a sanitizing agent, and a surface element coupled to a top surface of the sponge element, the surface element being permeable with respect to the sanitizing agent, thereby enabling the sanitizing agent to come into contact with a user's sole.

In another exemplary embodiment, the present invention is a method for sanitizing. The method includes impregnating a sponge element with a sanitizing agent, coupling a surface element to a top surface of the sponge element, placing the sponge element within a base portion, the base portion including a reservoir surrounded by side walls, the reservoir being sized to accommodate the sponge element, and filling the reservoir with a solvent to create a sanitizing solution that traverses the sponge and the surface element when pressure is applied.

### BRIEF DESCRIPTION OF THE DRAWINGS

FIG. 1 shows a sanitizing mat, according to an exemplary embodiment of the present invention.

FIGS. 2A-2C show a base portion of a sanitizing mat, according to an exemplary embodiment of the present invention.

FIG. 3 shows a sponge element and a surface element, according to an exemplary embodiment of the present invention.

FIGS. 4A and 4B show a plurality of surface elements on top of a sponge element, according to an exemplary embodiment of the present invention.

FIGS. 5A and 5B show a sanitizing mat including a blotting portion, according to an exemplary embodiment of the present invention.

FIGS. 6A and 6B show a sanitizing mat containing a scratching portion for use with pets, according to an exemplary embodiment of the present invention.

FIG. 7 shows a method of creating a sanitizing mat, according to an exemplary embodiment of the present invention.

### DETAILED DESCRIPTION OF THE INVENTION

The present invention provides an apparatus and methods for sanitizing the soles of footwear and feet. The apparatus comprises a sanitizing mat including a sponge element impregnated with a sanitizing agent, the sponge element being placed within a base portion having a reservoir adapted to fit the sponge portion. A surface element is coupled to the top surface of the sponge element. The sanitizing agent may be an aqueous solution. The surface element is composed of a permeable material, such that when a user steps on the sanitizing mat, the resultant pressure squeezes the sanitizing solution out of the sponge element and through the surface element to sanitize the feet or footwear of the user. The surface element may be an abrasive material, such that debris is brushed off from the user's feet or footwear and left on the surface element. The surface element may further be textured to facilitate the flow of the sanitizing solution towards the soles of the user's feet or footwear. The user then steps off of the mat without tracking germs into the house.

Embodiments of the present invention further include a blotting portion. The blotting portion can either be coupled to the sanitizing mat or is separate from the sanitizing mat. Stepping from the sanitizing mat to the blotting portion allows a user to dry excess sanitizing solution from the soles of his or her feet or footwear. The blotting portion may be folded onto or placed onto the sanitizing mat to reduce evaporation of the sanitizing solution.

A sanitizing solution as described herein and used in the invention includes any non-toxic solution that kills, destroys, or otherwise renders sterile a substantial portion of the microorganisms present on the surface that the sanitizing solution comes in contact with. In embodiments of the invention, the sanitizing solution is poured or sprayed onto the surface element, the sponge element, or the reservoir, forming a pool in the reservoir which may be absorbed into the sponge layer. As the solution evaporates, or is transferred to soles of shoes and feet, the solution may be ‘recharged’. In order to recharge the invention, the sanitizing solution is poured into or sprayed onto the reservoir, the surface element, and/or the sponge element. In embodiments of the invention, the solution falls into the category of generally safe and effective (GRASE). Examples of such a sanitizing solution are water-based solutions containing a sanitizing agent, such as benzalkonium chloride (BZK) and/or citric acid. Both of these substances are non-toxic, i.e., they are safe and effective for humans and pets. Alcohol-based solutions may also be used for the sanitizing solution, though these solutions tend to evaporate more quickly and would require recharging more frequently.

In alternative embodiments, the sanitizing agent may be impregnated or embedded into the sponge layer such that the sanitizing agent may be activated with water. Rather than recharging the invention with a sanitizing solution, a user simply must pour or spray water onto the mat and/or reservoir. The water combines with the embedded BZK and/or citric acid to form the sanitizing solution.

Embodiments of the sanitizing solution include a scent. For instance, the sanitizing solution may contain a fragrance, such as a “fresh scent” that spreads through a room as the sanitizing solution evaporates or is transported. Such a scent may be, for instance, catnip in order to cause pets to be attracted to and/or scratch a portion of the sanitizing mat.

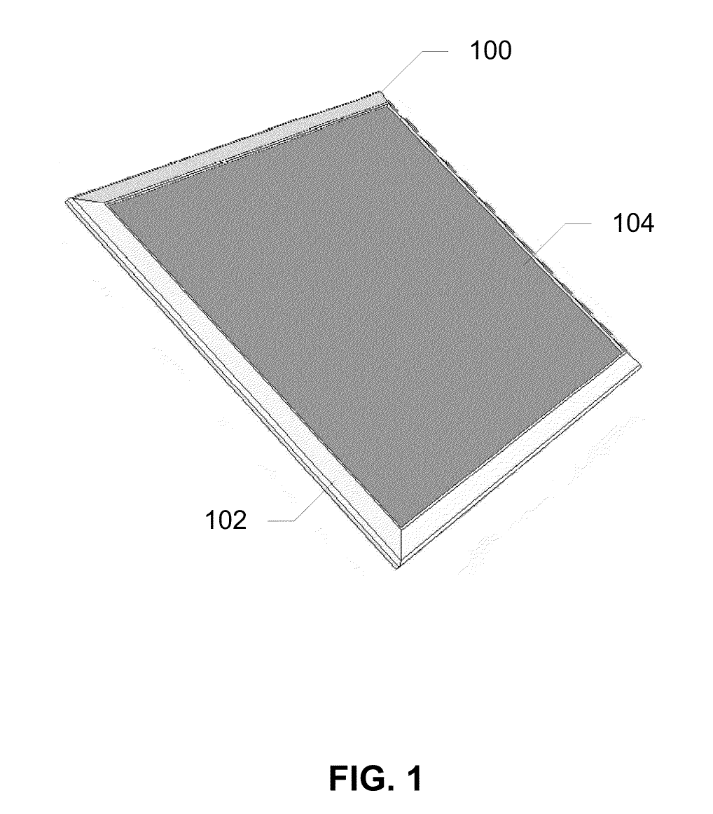

FIG. 1 shows a sanitizing mat 100, according to an exemplary embodiment of the present invention. In this embodiment, sanitizing mat 100 includes a base portion 102 enclosing a sponge element (not shown), and a surface element 104 above the top surface of the sponge element. The sponge element and surface element 104 are sized to fit within a reservoir formed within base portion 102. Any corner or edge of the sponge element and surface element 104 may be lifted from base portion 102 in order to add sanitizing solution to the reservoir. Alternatively, the sanitizing solution may be poured onto surface element 104 and absorbed into the sponge element, with the excess sanitizing solution trapped by and remaining within the reservoir. The sanitizing solution pools within the reservoir of base portion 102 from where it is soaked into the sponge element as needed. When a user steps on sanitizing mat 100, the resultant pressure squeezes the sanitizing solution contained within the sponge element through surface element 104 onto the sole of the user's foot or footwear. The user may wipe his or her soles on sanitizing mat 100, spreading the sanitizing solution and sanitizing the soles.

In embodiments of the present invention the base portion is a rubber or plastic base mat, such as a doormat tray. Due to the characteristics of rubber, such a base portion is resistant to slipping on a floor or other surface. In other embodiments of the invention, the base portion is formed of a different material, or a combination of materials that provides structure and is impermeable to fluids such as sanitizing solutions. Other materials which are not as slip-resistant as rubber may contain rubber feet, an adhesive, or other means of preventing slipping between the bottom of the base portion and the floor.

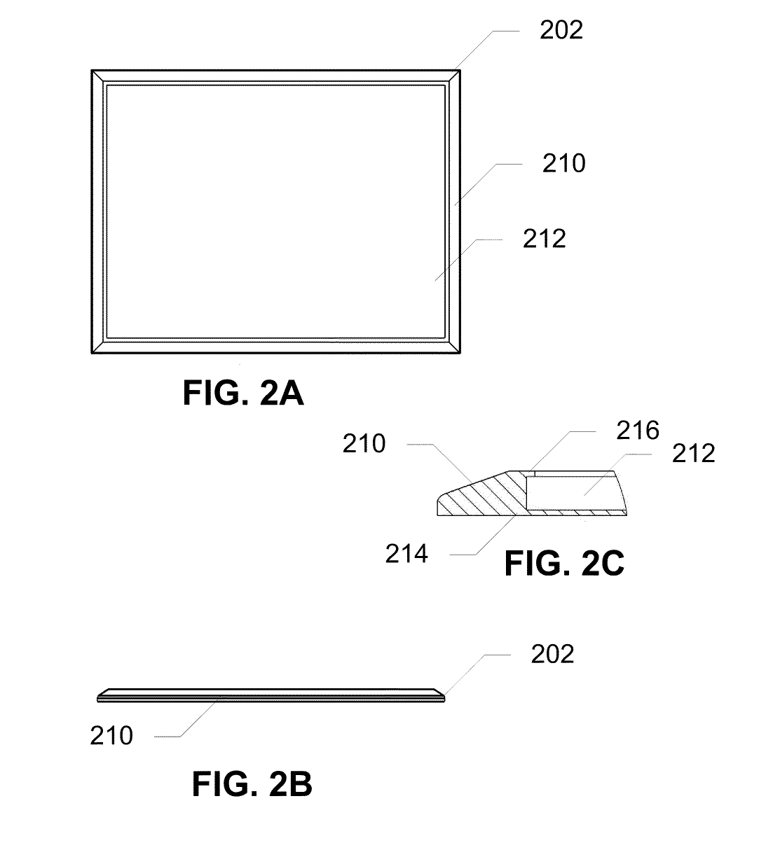

FIGS. 2A-2C show a base portion 202 of a sanitizing mat, according to an exemplary embodiment of the present invention. FIG. 2A shows an overhead view of base portion 202. Base portion 202 includes a reservoir 212 that is formed by a bottom surface and side walls 210 of base portion 202. FIG. 2B shows a side view of base portion 202. Side walls 210 of base portion 202 have a tapered edge around the outer edge of base portion 202 with the tapering ending at an inner wall of side wall 210 surrounding reservoir 212. Tapered edges ensure that base portion 202 is not easily kicked out of place when a user approaches the sanitizing mat. Reservoir 212 is sized such that the sponge element fits snugly within reservoir 212, allowing the sponge element to contact sanitizing solution within reservoir 212. Base portion 202 adds structure to the sanitizing mat and keeps the sanitizing solution in or around the sponge element by creating a pooling effect.

FIG. 2C shows a cutaway side view of base portion 202. The inner surface of side wall 210 of the base portion rises at a right angle from reservoir 212. A lip 216 around the upper edge of reservoir 212 extends from the side wall 210 at a right angle to a point above reservoir 212. Lip 216 keeps the sponge element and/or surface element in place within reservoir 212. Thus, the sponge element and/or the surface element are prevented from sliding within base portion 202 or out of base portion 202 when a user is wiping his or her feet on the sanitizing mat. Lip 216 is of any length that provides this functionality while allowing for the removal of sponge element and surface element when needed. Lip 216 is designed to be flexible in order to accomplish the addition or removal of the sponge element and/or surface element.

Further embodiments of the present invention include other means to keep the sponge element and/or surface element in place. These means can include clips, fasteners, Velcro strips, hooks, etc. These may be spaced around the outer edges of the sponge element and/or the surface element to couple the element(s) to the base portion.

According to embodiments of the invention, the reservoir is a ½ inch reservoir where the side walls of the reservoir are ½ inch tall to enclose the sides of a ½ inch thick sponge element.

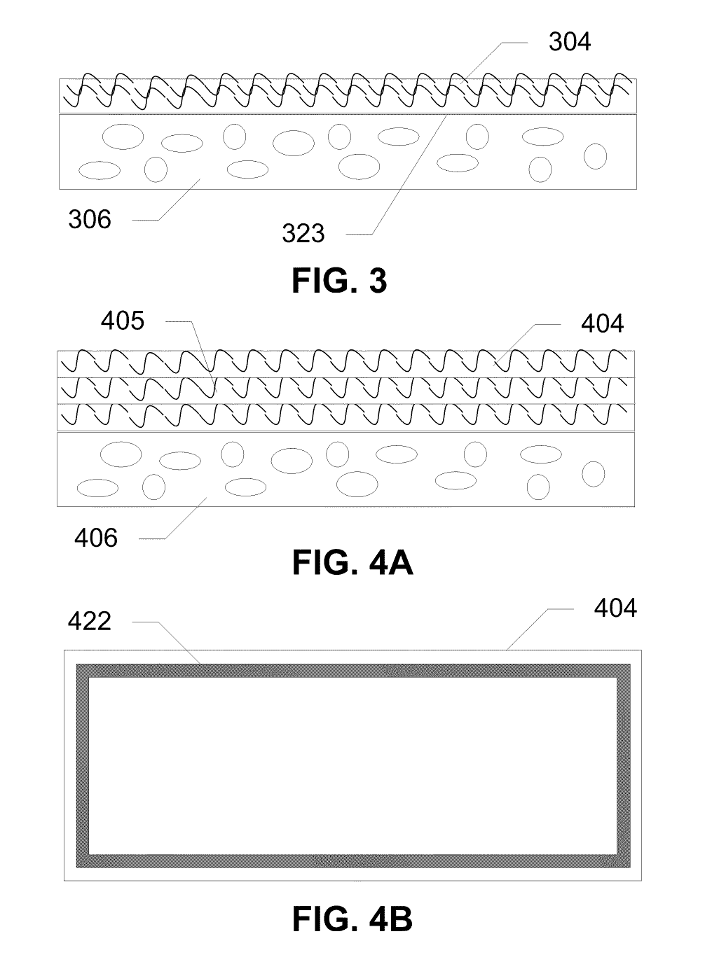

FIG. 3 shows a sponge element 306 and a surface element 304, according to an exemplary embodiment of the present invention. In this embodiment, sponge element 306 and surface element 304 are bonded together at bond 323 through conventional techniques. Sponge element 306 is impregnated with a sanitizing agent or a sanitizing solution. Excess solution is retained by the base portion of the sanitizing mat as described above. This allows the sanitizing solution to seep out of sponge element 306 when the sanitizing mat is stepped on, but soak up excess sanitizing solution such that the sanitizing solution is not constantly on the upper surface of surface element 304. Sponge element 306 may be any other porous material with absorbing properties, including but not limited to cellulose wood fibers, foamed plastic polymers, or some combination of materials. Synthetic forms of sponges used may be low-density polyether, polyvinyl alcohol or “PVA” (a very dense, highly absorbent material with no visible pores), polyester, etc. Sponge element 306 may have small or closed cells to absorb more fluid, although any type of sponge or sponge-like material may be used.

Surface element 304 of the present invention is composed of a material or materials that are relatively non-absorbent, but that attract dirt and other particles. Surface element 304 is permeable, allowing the sanitizing solution to pass through from sponge element 306 to the user's feet or footwear while absorbing little of the sanitizing solution. Surface element 304 is affixed or bonded to sponge element 306 at bond 323, and comprises an abrasive material. This abrasive material may be, for example, a plastic mesh, a coarse foam, a coarse polymer based material, etc. According to embodiments of the present invention, the surface element is a layer of polypropylene. Polypropylene is a thermoplastic polymer, made by the chemical industry and used in a wide variety of applications. Polypropylene is rugged and unusually resistant to many chemical solvents, bases and acids. The use of polypropylene generally decreases the evaporation of the antimicrobial solution. Surface element 304 may also simply be a textured cloth or paper fiber based material. The abrasive material or texture penetrates small cavities in the sole of a user's footwear, dislodging particles from the soles of the user's feet or footwear. While the user is wiping his or her feet, the sanitizing solution seeps up from sponge element 306, through surface element 304, to sanitize the user's feet or footwear. Surface element 304 also serves to spread the sanitizing solution on the sole of the foot or footwear more effectively.

A combined surface element 304 and sponge element 306 undergoes some wear, and therefore can be replaced when degraded or dirty. The combined surface element 304 and sponge element 306 is simply removed from the base portion and a new combined surface element and sponge element is placed into the base portion. In alternative embodiments of the present invention, the surface element may be reusable. In these embodiments, for instance, the surface element, the sponge element, or the surface element and sponge element combination may be machine-washable such that it can be removed, washed, and placed back into the base portion of the sanitizing mat.

In further embodiments of the present invention, the surface element is not bonded to the sponge element, and may be replaced separately from the sponge element. In such embodiments, a plurality of surface elements is attached to the base portion above the sponge element and/or clipped to the sponge element. The surface elements may be replaced as desired or as needed due to staining and debris. Exemplary embodiments of the invention utilize, for example, five to ten surface element layers in a manner such that the topmost surface element may be disposed of, exposing a new fresh surface element still in place. In this way, used surface elements get disposed of as needed, by disengaging the top surface element, thereby exposing a fresh surface element.

FIGS. 4A and 4B show a plurality of surface elements 405 on top of a sponge element 406, according to an exemplary embodiment of the present invention. FIG. 4A shows plurality of surface elements 405 layered one on top of the other with an adhesive that allows a top surface element 404 to be peeled off from plurality of surface elements 405. As seen in FIG. 4B, this is accomplished using an adhesive strip 422 surrounding the outer edge of a bottom surface of surface element 404. Each surface element of plurality of surface elements 405 adheres to the surface element beneath it in a similar manner. Adhesive strip 422 attaches each of plurality of surface elements 405 at a strength such that top surface element 404 may be removed with relatively little force, but stays in place during regular use of the sanitizing mat.

In certain embodiments of the present invention, the adhesive may also cover the entire bottom of each surface element, with the adhesive at a strength that allows the top surface element to be removed. According to embodiments of the present invention, the surface elements include tabs that may be grabbed and pulled by the user to easily remove the top surface element. These tabs extend from the surface element and may be tucked away into the base portion when not needed. Alternatively, the tabs may be an area at a corner of the bottom of the surface element not containing the adhesive such that the area is more easily pulled back.

In further embodiments of the present invention, the surface elements may be otherwise affixed to each other. According to embodiments of the invention, the surface elements may be affixed to each other by a static charge. This allows the surface elements to stick to each other, without the use of adhesive. These embodiments may also contain tabs to make the removal of the top surface element easier. In further embodiments, the surface elements are affixed together by fasteners such as clips. The clips may be disengaged to remove the top surface element and then re-engaged to secure the remaining surface elements.

Embodiments of the surface element may contain designs and writing. These designs and writing may be customizable or may be one of various conventional “welcome” mat designs currently used. The designs may comprise raised portions of the surface layer, creating an abrasive region. This raised portion may be composed of the same material as the rest of the surface elements or may be a different material bonded to the surface layer.

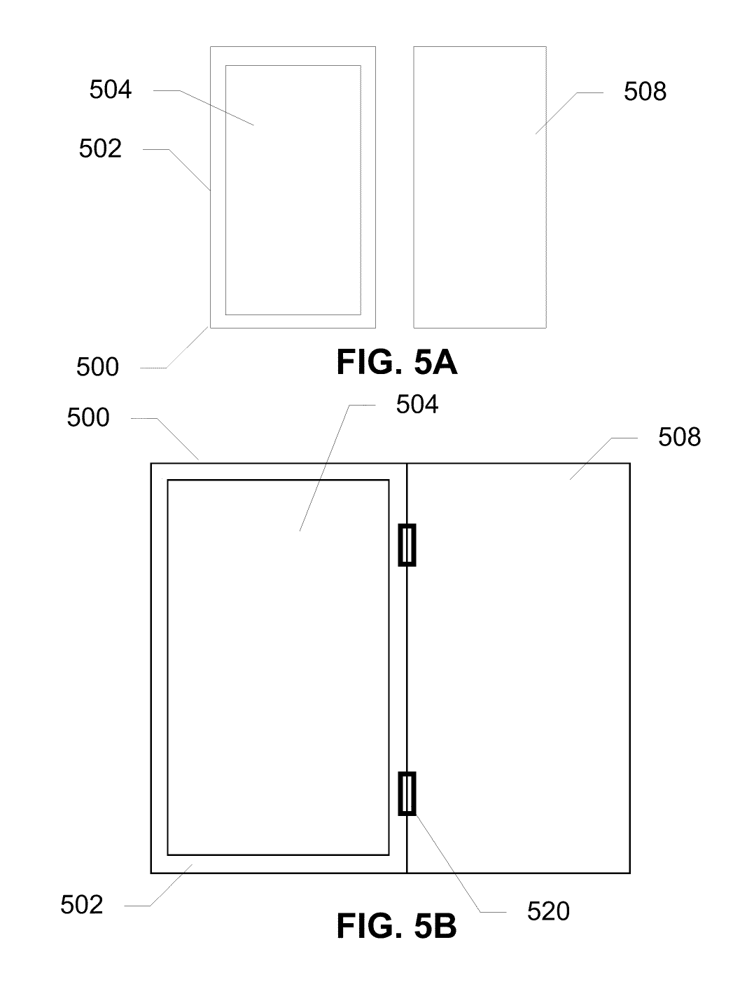

FIGS. 5A and 5B show a sanitizing mat 500 including a blotting portion 508, according to an exemplary embodiment of the present invention. Blotting portion 508 allows a user to dry any excess sanitizing solution off of the soles of his or her footwear or feet before entering the rest of the room. As shown in FIG. 5A, blotting portion 508 is a pad of absorbent material placed adjacent to sanitizing mat 500. Blotting portion 508 may be any absorbent material or combinations of materials including but not limited to fabric, foam, another sponge-like material, etc. Blotting portion 508 may be a similar size as sanitizing mat 500 or larger or smaller. The bottom surface of blotting portion 508 is preferably a sticky or grippy material, for instance, like the rubber used in base portion 502. The bottom surface helps prevent blotting portion 508 from slipping during use. In addition, blotting portion 508 may be placed over surface element 504, covering surface element 504 when sanitizing mat 500 is not in use. In this state, the rubber bottom of blotting portion 508 reduces the evaporation of sanitizing solution from sanitizing mat 500.

FIG. 5B shows a blotting portion 508 coupled to a sanitizing mat 500, according to an exemplary embodiment of the present invention. In this embodiment, blotting portion 508 is coupled to base portion 502 of sanitizing mat 500 using a plurality of hinges 520 or equivalent connectors. Hinge 520 allows blotting portion 508 to flip over surface element 504. In a closed state, this reduces evaporation of the sanitizing solution and reduces the overall floor space taken by sanitizing mat 500. In an open state, blotting portion 508 enables a user to easily discard excess solution from the soles of his or her feet. Hinge 520 also serves to prevent blotting portion 508 from moving too far away from sanitizing mat 500.

In further embodiments of the present invention, the blotting portion coupled to the sanitizing mat includes a rigid tab or protrusion enabling a user to flip the blotting portion open to use it, and flip the blotting portion back, simply using his or her feet. In further embodiments of the present invention, the blotting portion may also be a portion of the sanitizing mat, such that one steps off of the portion of the mat where the surface element is located and onto the blotting portion.

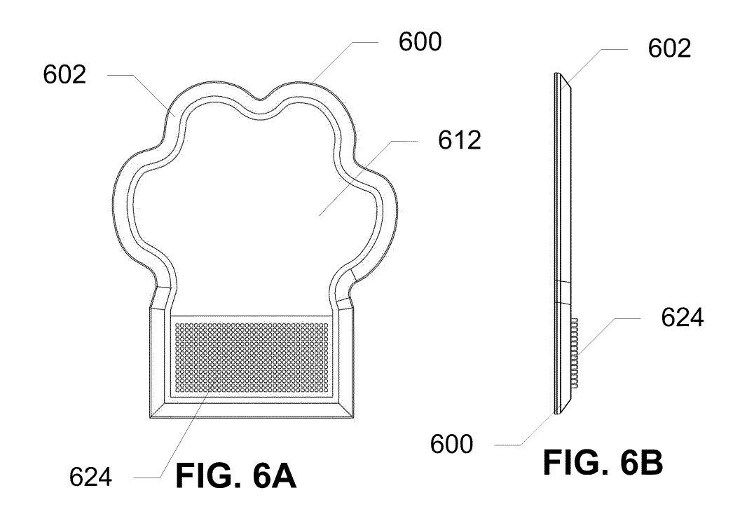

FIGS. 6A and 6B show a sanitizing mat 600 containing a scratching portion 624 for use with pets, according to an exemplary embodiment of the present invention. According to this embodiment of the present invention, sanitizing mat 600 includes a base portion 602, a reservoir 612, a sponge element (not shown), a surface element (not shown), and scratching portion 624. Although base portion 602 is shown in a paw shape, this is purely ornamental, and base portion 602 may take any shape that includes a bottom surface and a side wall to create reservoir 612. Reservoir 612 similarly holds the sponge element that is appropriately sized to fit the reservoir, along with the surface element, and the sanitizing solution. Scratching portion 624 is abrasive enough such that pets may scratch scratching portion 624 to clean and file down nails. Scratching portion 624 is ideally a sandpaper-like material functional to remove particles from the nails as well as file the nails down, similar to a common nail file, and deteriorates with use.

According to embodiments of the present invention, the scratching portion may be a portion of the surface element, may be the entire surface element, or may be located adjacent to the surface element on the base portion. The scratching portion may be comprised of any material that has the effect of filing down the nails of pets.

FIG. 6B shows a scratching portion 624 on a raised area of sanitizing mat 600, according to an exemplary embodiment of the present invention. Raising scratching portion 624 with respect to the rest of sanitizing mat 600 may entice pets to scratch this area. Having scratching portion 624 separate from the surface element allows scratching portion 624 to be replaced independently of the surface element. This may prove useful as scratching portion 624 degrades as it is used by pets.

In order to attract pets to a scratching portion, catnip or any other attractive smelling substance may be used. The substance is combined into the sanitizing agent, such that the attractive odor is recharged along with the sanitizing solution when a solvent such as water is poured onto the mat. The catnip should be placed such that it draws pets to the scratching portion. The catnip may be dispersed between the sponge element and surface element, between surface elements, beneath the scratching portion, impregnated into the surface element, scratching portion, or sponge element, etc., depending on the placement of the scratching portion. The catnip, along with a degradable scratching portion, causes pets, such as cats, to use the scratching portion for scratching.

According to embodiments of the invention, other scents, such as deodorizers, may be combined with the sanitizing solution. Such scents are transferred to the environment of the sanitizing mat as the sanitizing solution evaporates or is otherwise spread. These scents may also be embedded within the sponge element or the surface element. In embodiments of the present invention with a plurality of surface elements, pulling off the top surface element may activate a scent on the surface element below. Thus, as the top surface element is removed, the next surface element becomes the top surface element and has a newly activated fresh scent.

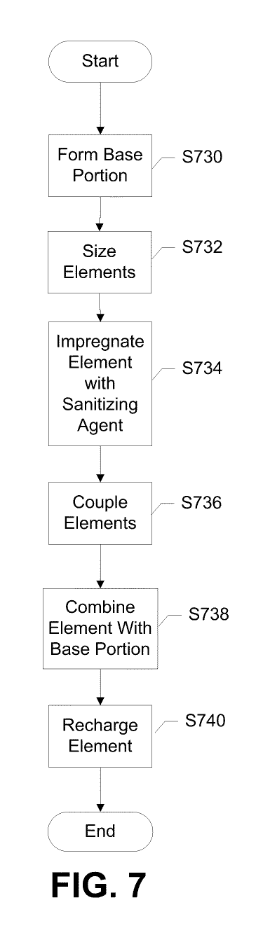

FIG. 7 shows a method of creating a sanitizing mat, according to an exemplary embodiment of the present invention. In this embodiment, a base portion is formed from rubber or other material S730. The base portion is formed in a desired shape having a floor or bottom surface with raised side walls to form a reservoir. A surface element and sponge element are sized to fit together within the reservoir of the base portion S732. This may be accomplished through forming the materials, cutting the materials, etc. One or both of the sponge element and surface element is impregnated with a sanitizing agent S734. This sanitizing agent may be, for example, dried citric acid or BZK. The surface element and sponge element are coupled or bonded together S736. This may be accomplished using an adhesive, by heat bonding, etc. The steps of sizing the elements, impregnating the elements, and bonding the elements may take place in any order, as the elements may be bonded and impregnated before sizing, impregnated then bonded before sizing, etc. The surface element/sponge element combination is further combined with the base portion S738. This is accomplished by placing the combined elements into the reservoir of the base portion. The combined elements may then be secured into the base portion by folding the combined elements under a lip of the base portion. Alternatively, the combined elements may be otherwise clipped or attached to the base portion. The combined elements are then recharged S740. This is accomplished by adding water or sanitizing solution to the reservoir of the base portion. Water activates the impregnated sanitizing agent to create the sanitizing solution. The sanitizing mat may then be placed at a location and used to sanitize feet and footwear.

In other embodiments of the present invention, the sponge portion is pre-impregnated with a sanitizing solution, coupled with the surface portion, and provided in a sealed container. A user would remove the sealed container or cover and place the sponge portion within the reservoir. In other embodiments of the present invention, there may also be a bristle portion of the mat. This allows one to brush off loose dirt and other particles before stepping onto the sanitizing portion of the mat. This may allow each surface element layer to be used longer, as the surface element does not collect as much of this dirt.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. An apparatus for sanitizing, the apparatus comprising:
a base portion having a reservoir, the reservoir formed by a bottom surface of the base portion and a plurality of side walls of the base portion;
a sponge element coupled to the base portion, the sponge element being impregnated with a sanitizing agent; and
a surface element coupled to a top surface of the sponge element, the surface element being permeable with respect to the sanitizing agent, thereby enabling the sanitizing agent to come into contact with a user's sole.

2. The apparatus of claim 1, wherein the surface element is bonded to the top surface of the sponge element and has a textured surface.

3. The apparatus of claim 1, further comprising a plurality of surface elements coupled to the top surface of the sponge element, wherein the plurality of surface elements are attached to each other with adhesive strips.

4. The apparatus of claim 1, wherein the side walls of the base portion include a lip protruding over a portion of the bottom surface to secure a portion of the top surface of the sponge element.

5. The apparatus of claim 1, further comprising a means to secure the sponge element to the base portion.

6. The apparatus of claim 1, wherein the sanitizing agent contains one of citric acid and benzalkonium chloride.

7. The apparatus of claim 1, further comprising a blotting portion coupled to the base portion, the blotting portion comprising a rubber sheet bonded to an absorbent material.

8. The apparatus of claim 7, wherein the blotting portion is coupled to the base portion via a hinge.

9. The apparatus of claim 1, further comprising a scratching portion coupled to the base portion, the scratching portion comprising an abrasive material.

10. The apparatus of claim 1, further comprising a scent embedded within one of the sponge element, the surface element, and the sanitizing agent.

11. The apparatus of claim 1, wherein the sanitizing agent creates an aqueous solution when water is added to the reservoir.

12. A method for sanitizing, comprising:
impregnating a sponge element with a sanitizing agent;
coupling a surface element to a top surface of the sponge element;
placing the sponge element within a base portion, the base portion including a reservoir surrounded by side walls, the reservoir being sized to accommodate the sponge element; and
filling the reservoir with a solvent to create a sanitizing solution that traverses the sponge and the surface element when pressure is applied.

13. The method of claim 12, further comprising coupling a plurality of sponge elements to the surface element.

14. The method of claim 13, wherein the plurality of sponge elements are removably attached to each other with an adhesive.

15. The method of claim 12, further comprising recharging the reservoir with the solvent to create more sanitizing solution.

16. The method of claim 12, wherein the sanitizing agent is one of citric acid and benzalkonium chloride.

17. The method of claim 12, wherein the base portion is formed out of rubber.

18. The method of claim 12, further comprising coupling a blotting portion to the base portion.

19. The method of claim 18, wherein the blotting portion comprises a rubber sheet bonded to an absorbent material.

20. The method of claim 12, further comprising adding a scent to one or more of the sponge element, the surface element, and the sanitizing solution.
